(19) United States
(12) Patent Application Publication
(10) Pub. No.: US 2026/0139333 A1
(43) Pub. Date: May 21, 2026

(54) QPCR METHOD AND KIT HAVING INCREASED SPECIFICITY AND SENSITIVITY

(71) Applicant: GENOTECH CORP., Daejeon (KR)

(72) Inventors: Jae Jong KIM, Daejeon (KR); Si-Kyu LIM, Gyeonggi-do (KR); Ayoung KYUNG, Daejeon (KR)

(21) Appl. No.: 18/869,444

(22) PCT Filed: Jun. 22, 2023
(86) PCT No.: PCT/KR2023/008683

§ 371 Date: Nov. 26, 2024

(30) Foreign Application Priority Data
Jul. 12, 2022 (KR) 10-2022-0085659
Jun. 22, 2023 (KR) 10-2023-0080355

(51) Int. Cl. C12Q 1/70; C12Q 1/6851

(52) U.S. Cl. CPC C12Q 1/701; C12Q 1/6851; C12Q 2600/156

(57) ABSTRACT

A method for adding a com probe which is a non-amplified non-fluorescent probe through a method for controlling non-specific signals generated by the non-specific binding of fluorescent probes occurring in a high-concentration wild-type sample. The method for adding a com probe or a PCR kit capable of performing the method, serves to inhibit non-specific signals generated by the binding of a fluorescent probe to a wild-type nucleic acid sequence, so as to enhance the specificity and positive detection of amplification signals of a mutant nucleic acid sequence, and thus can increase detection reliability.

### CROSS REFERENCE TO RELATED APPLICATIONS

This application is a National Stage of International Application No. PCT/KR2023/008683 filed Jun. 22, 2023, claiming priority based on Korean Patent Application No. 10-2022-0085659 filed on Jul. 12, 2022 and Korean Patent Application No. 10-2023-0080355 filed on Jun. 22, 2023.

### SEQUENCE LISTING

The instant application contains a Sequence Listing which has been submitted via Patent Center and is hereby incorporated by reference in its entirety. Said .xml copy, created on Dec. 9, 2025 is named Q316099SequenceListing12092025.xml, and is 35,869 bytes in size.

### TECHNICAL FIELD

The present invention relates to a method and a kit for increasing the specificity and sensitivity of real-time polymerase chain reaction (PCR) for distinguishing and detecting specific genetic variants, and more specifically, the present invention relates to a quantitative PCR (qPCR) method and kit that increase the positive detection rate and detection specificity of a variant nucleic acid by inhibiting the amplification of a wild-type (non-variant) nucleic acid sequence by fluorescent probes used to detect variants, and the present invention increases the accuracy and sensitivity of distinguishing a variant nucleic acid sequence and a wild-type nucleic acid sequence, thereby allowing variants to be distinguished or detected more easily.

### BACKGROUND ART

A common method for distinguishing nucleic acid sequence variations is quantitative real time PCR (qPCR), which applies the following molecular diagnostic method to test single nucleotide polymorphisms (SNPs). The qPCR method is the most widely used, is fast, has high sensitivity and specificity, has low analysis costs, and has the advantage of being easily automated.

The qPCR method is known as an analysis method using TaqMan Probe, a type of hydrolysis probe, and the basic principle applied is to use the 5′→3′ exonuclease activity of a Taq DNA polymerase (Holland P. M. et al., 1991. Proc. Natl. Acad. Sci., 88:7276-7280). In 1991, Holland et al. showed that when a probe having a base sequence complementary to template DNA is used, a specific PCR reaction could be confirmed in real-time by the 5′→3′ exonuclease activity of the Taq DNA polymerase. Since then, based on this method, various qPCR techniques using probes modified with fluorescent pigments have been developed and are widely used in various fields (Heid, C. A. et al., 1996. Genome Res. 6. 986-994.: Livak K. J. 1999. Genet. Anal., 14:143-149).

The method of using a hydrolysis probe uses a probe with a reporter and a quencher bound to both ends along with a primer during the PCR reaction, and it uses the principle of fluorescence resonance energy transfer. In other words, when the reporter and the quencher are adjacent, the energy released from the reporter is transferred to the adjacent quencher and no fluorescence is detected, but as the PCR amplified product increases, the probe bound to the target gene is hydrolyzed by the 5′→3′ nuclease activity of the Taq DNA polymerase, and fluorescence of the reporter is emitted. In the TaqMan probe assay, which is an analysis method using a hydrolysis probe, it is very important to find the optimal conditions under which the probe may bind to the target base sequence and subsequently be hydrolyzed by nuclease activity. In other words, PCR conditions (thermal profile) that simultaneously allow the primers and probes used in PCR to hybridize to the target sequence and the probes to be hydrolyzed are important. To meet these two requirements, two-step PCR is usually applied. In other words, a denaturation step is performed at 95° C., and extension is performed after annealing at a temperature 7 to 10° C. lower than the Tm of the probe. When the second step reaction is performed at an excessively high temperature, the probe will be separated from the target (strand displacement) rather than hydrolyzed by the 5′→3′ nuclease activity of the Taq DNA polymerase, and fluorescence will not increase (Logan, J. et al. 2009. Caister Academic Press). The TaqMan™ (dual labeled) probe, which distinguishes a non-variant sequence and a variant sequence by directly binding to a variant site, has the advantage of being able to detect single nucleotide polymorphisms (SNPs) or analyzing mutations using various fluorescent substances. However, in this method, a short probe must be used to provide specificity to the probe, which inevitably lowers the Tm value, making it difficult to maintain a stable annealing state.

To overcome the above shortcomings, there is a disadvantage that expensive minor groove binder (MGB) probes or locked nucleic acid (LNA) probes must be used (Letertre, C. et al. 2003. Mol. Cell Probes, 17:307-311). The MGB TaqMan probe is similar to a regular TaqMan probe, but as a minor groove binding portion is added to the 3′ end, the Tm is high despite the short probe length, allowing it to maintain a stable annealing state under PCR conditions (Kutyavin, I. V. et al. 2000. Nucleic Acids Res., 28:655-661).

Instead of using a separate modified probe like MGB, SNPs are analyzed by applying the amplification-refractory mutation system (ARMS) PCR principle with a TaqMan probe (Ellison, G. et al. 2010. J. Exp. Clin. Cancer Res., 29:132). However, in the ARMS PCR method, it is very difficult to find the optimal PCR conditions to distinguish SNPs (Punia P. and Saunders. N. www.horizonpress.com/pcrbooks). In addition, when a large amount of wild-type genes are included during ARMS PCR, many false positive detections frequently occur.

SARS-COV-2 (Covid-19) is an RNA virus, and mutations in its genetic sequence occur during replication in the body of animals such as humans. These sequence mutations lead to the emergence of clinically important variants such as the delta variant (B.1.617.2), omicron variant (BA.1), and stealth omicron variant (BA.2). The above variants are important in terms of public health because of their increased infectivity or increased pathogenicity. In the case of a pandemic or endemic, the emergence of new variants is becoming more frequent, and monitoring these variants is necessary for health epidemiological research on the frequency and timing and distribution of the emergence of variants, and it may also provide very important data to understand the fatality rate and transmissibility of each variant.

Accordingly, world health authorities, including the World Health Organization (WHO), classify and manage the variants by classifying groups of similar genetic variants into lineages or lineage groups and designating them as variants of concern (VOC) or variants of interest (VOI).

Depending on the lineages of the variants, the therapeutic effects of vaccines and antibody treatments may be reduced, and the sensitivity of antigen tests or PCR tests for monitoring infection and treatment may also be reduced. Accordingly, health authorities and medical sites need an effective and economical method to distinguish and detect variants.

In the case of viruses such as SARS-COV-2, the viral load in clinical samples is reported to vary from 3 to 10 log copies/ml depending on the progression of the patient's disease (D. Jacot, G. Greub, K. Jaton et al., doi.org/10.1016/j.micinf.2020.08.004).

A high viral load may be a factor that facilitates diagnosis because a low Ct value is exhibited when detecting infection using PCR, but when diagnosing a variant, a high viral load may cause false positives due to non-specific detection.

The threshold may be increased due to the amount of contamination that contributes to non-specific detection, and non-variant wild-type sequences may act as a contaminant that interferes with variant-specific detection. Therefore, a high wild-type (non-variant) viral load generates non-specific fluorescent signals, increasing the possibility of false positives.

PCR methods for variant detection include methods using sequence-specific primers such as allele-specific PCR (AS-PCR) and amplification-refractory mutation system PCR (ARMS-PCR). However, these methods have a high probability of non-specific amplification of the 3′ end of the primer, especially in the case of a high viral load, making it difficult to distinguish variants. Typically, variant differentiation is 0.1% or higher, so false positives may occur when the wild-type viral load is more than 105 copies/reaction.

Another PCR method for detecting a variant is a method using hydrolysis probes that may distinguish mutation regions. To distinguish variants using this method, control of the Tm value of the hydrolysis probes is very important. In general, although the use of a fluorescent hydrolysis probe (hydrolysis probe, TaqMan probe, dual labeled probe) having a high Tm value increases detection sensitivity by increasing the fluorescence signal value due to hydrolysis, such as relative fluorescence unit (RFU), when the viral load of a non-variant sample is high, non-specific binding increases, thereby increasing the possibility of false positives. On the other hand, when a hydrolysis probe having a low Tm value is used, non-specific binding due to non-variant samples may be inhibited, but the possibility of false negatives in samples having a low viral load increases.

To solve the above problems, methods of setting the length of a probe having an appropriate Tm value or finely adjusting the temperature conditions of PCR are applied, but it is not easy to overcome the above problems. In addition, as a way to overcome the above problems, MGB probes having a relatively short sequence may be applied, but the cost is quite expensive.

As described above, there are still technical limitations in methods that may distinguish variants from clinical samples having various viral loads while exhibiting sufficient sensitivity and specificity.

### DISCLOSURE

### Technical Problem

One object of the present invention is to provide a polymerase chain reaction (PCR) kit and a PCR method that may easily distinguish variants from clinical samples with various viral loads while exhibiting sufficient sensitivity and specificity.

In particular, another object of the present invention is to provide a PCR kit and a PCR method that may distinguish trace variants that are present together with wild-type genes that are present in a large quantity with high accuracy.

### Technical Solution

The present inventors added a non-fluorescent competition probe (hereinafter referred to as a Com probe) corresponding to a fluorescent detection probe for distinguishing variants to a PCR solution, thereby inventing a method of economically implementing variant detection PCR having high sensitivity and high specificity, which is the purpose of the present invention. The term “Com probe” implies that it is hybridized with a non-variant target base sequence by competing with a fluorescent detection probe.

In real-time PCR to confirm the presence of variants in a template including a target base sequence in which a position with a high probability of mutation is known, the present invention uses a fluorescent hydrolysis probe for distinguishing target base sequence variants and a Com probe for inhibiting the non-specific signals of non-variant (wild-type) target base sequences.

In the present invention, the sequence of a fluorescent hydrolysis probe is identical to a variant target base sequence and differs from a non-variant target base sequence by one or more amino acids, preferably one to three, more preferably one or two amino acids. Generally, when a probe has one or more mismatches with a template, the robustness of hybridization is weak, and during the polymerization process of the DNA polymerase, the probe is displaced before hydrolysis by the 3′ nuclease of the enzyme occurs. However, when an excessive amount of non-variant template is present, a small amount of non-displaced fluorescent probe is hydrolyzed and a non-specific signal is generated (see FIG. 4A). The present invention provides a Com probe whose sequence matches the non-variant template to inhibit the generation of non-specific signals generated from an excessive amount of non-variant template. At this time, the Com probe does not contain a fluorescent material and is not connected to the fluorescent material. Since a Com probe does not undergo displacement after binding to a non-variant template (wild type, WT) that is present in an excessive amount in a sample, the generation of non-specific signals caused by a fluorescent hydrolysis probe may be inhibited. In addition, since the added Com probe mismatches the sequence of the variant, the Com probe has no effect on the hydrolysis of the fluorescent hydrolysis probe that matches the variant sequence, making variant detection by qPCR easy (FIG. 4B).

As described above, the method of adding the Com probe of the present invention to qPCR may provide an effect of inhibiting the non-specific signal values (reducing thresholds) appearing in high-concentration non-variant (wild type) samples that may result from the use of a high-Tm variant-specific fluorescent probe.

The present invention provides a method of introducing a non-fluorescent Com probe that has a sequence that matches the non-variant (wild type) template and mismatches a mutation region of a variant template, thereby providing a method for increasing positive detection of variants and increasing specificity. The method of the present invention is hereinafter referred to as “SNP Typing with excellent specificity II (STexS II).”

### Advantageous Effects

When using the method or kit of the present invention, the addition of a Com probe can achieve the effect of controlling high non-specific signals caused by a high-concentration wild-type sample in a qPCR test for variant detection.

The method or kit of the present invention addresses the issue of low positive detection due to a high threshold that occurs when a fluorescent hydrolysis probe having a high Tm value is used, by adding a Com probe to qPCR, so when a fluorescent hydrolysis probe having a high Tm value is used, the present invention strengthens the advantages of qPCR, which are a low Ct value, a small Ct value deviation, and high signals, and lowers the high threshold, which is a disadvantage, thereby increasing positive detection.

### DESCRIPTION OF DRAWINGS

FIG. 1 shows the effect on qPCR according to changes in the length (Tm value) of the fluorescent hydrolysis probe for variant detection.

As fluorescent hydrolysis probes, FAM-Cov19-Q954H(a), FAM-Cov19-Q954H-1(b), and FAM-Cov19-Q954H-3(c) were used to detect Q954H, JOE-Cov19-T547K-1(d), JOE-Cov19-T547K-3(e), and JOE-Cov19-T547K-5(f) were used to detect T547K, and FAM-Cov19-T19R-S1(g), FAM-Cov19-T19R-S2(h), and FAM-Cov19-T19R-S3(i) were used to detect T19R. A standard plasmid was used as a template, -Δ- denotes a variant test group with a copy number of 5×10³, -o- denotes a variant test group with a copy number of 5×10², and -□- denotes a variant test group with a copy number of 5×10¹, -⋄- denotes a non-variant wild-type control group with a copy number of 5×10⁷, and -X- denotes a template-free control group.

FIG. 2 shows the results of testing the degree of non-specific PCR signal inhibition according on the amount of Com probe added.

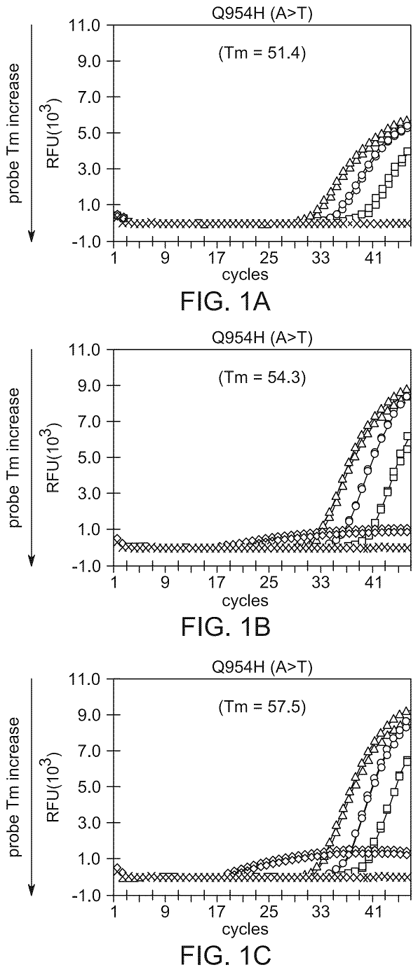

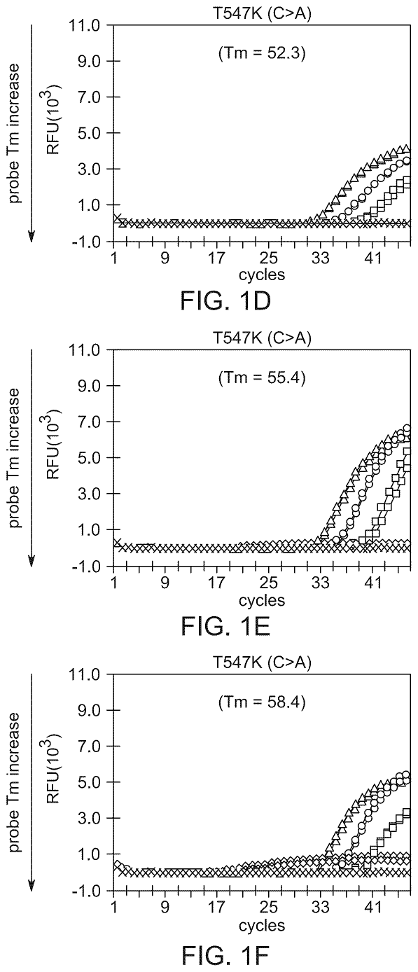

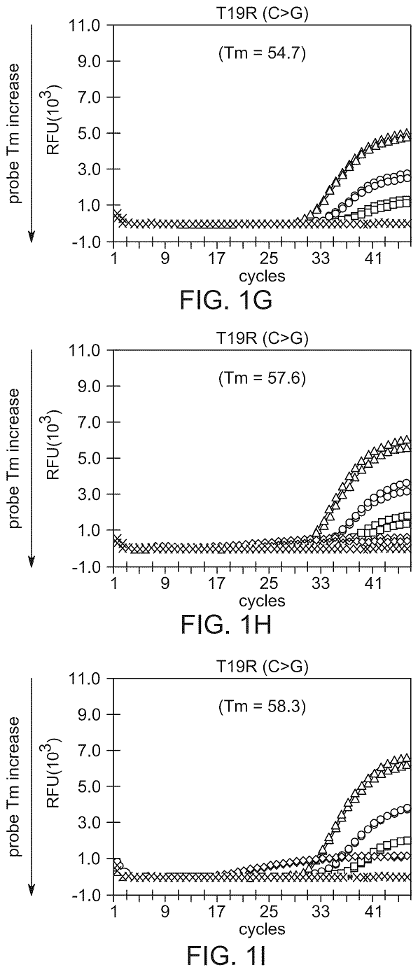

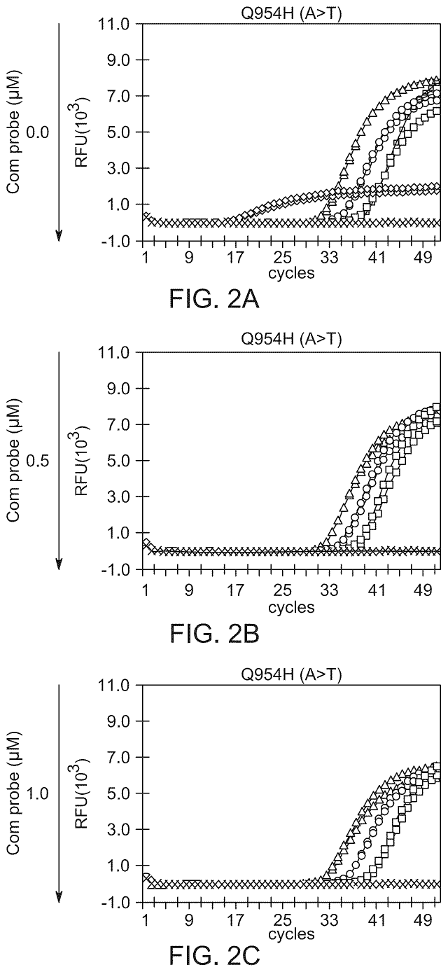

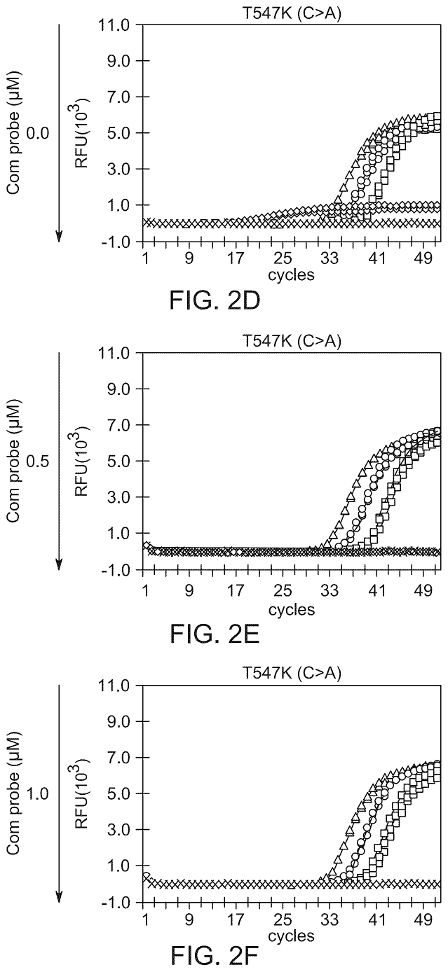

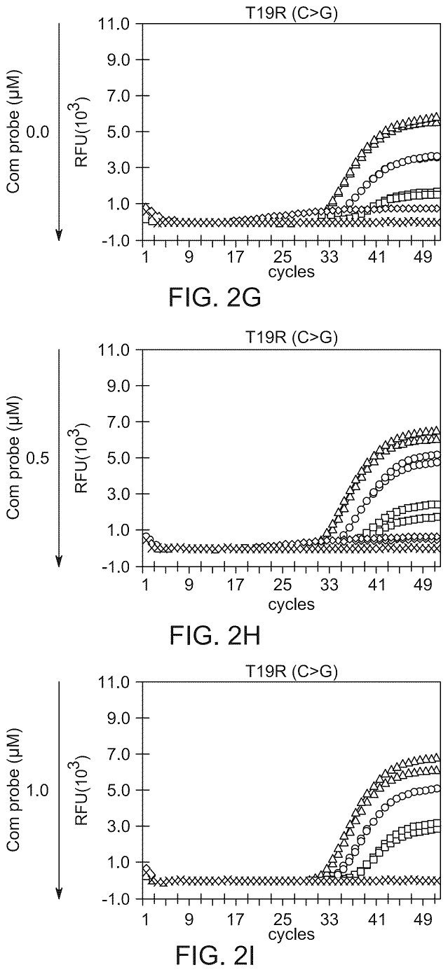

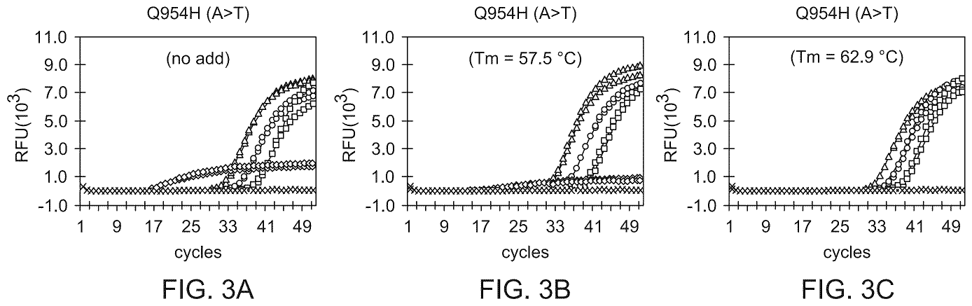

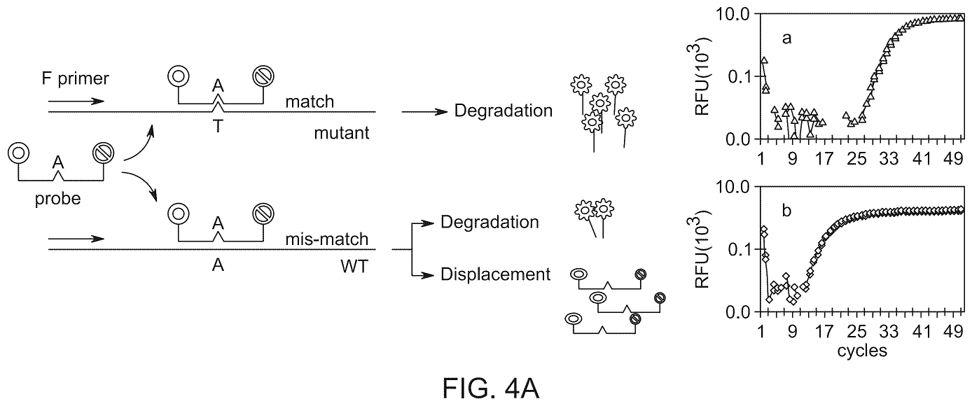

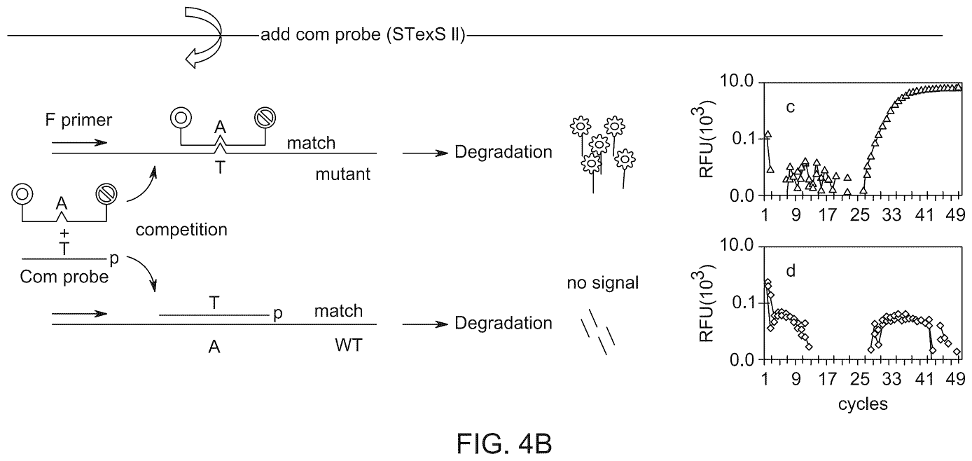

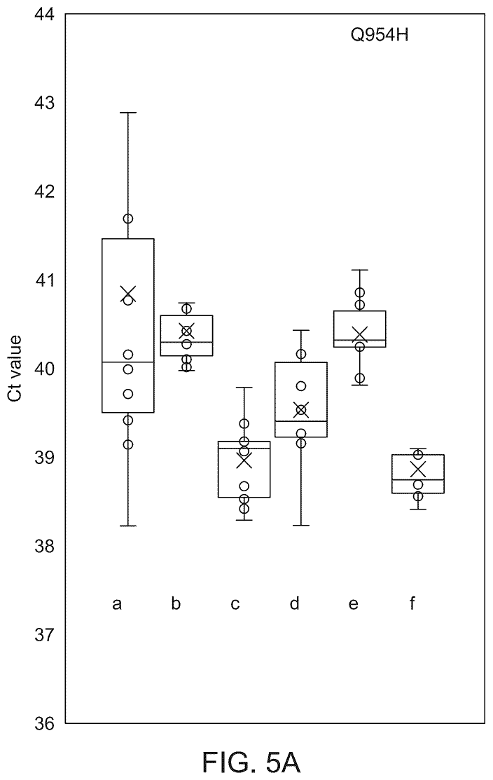

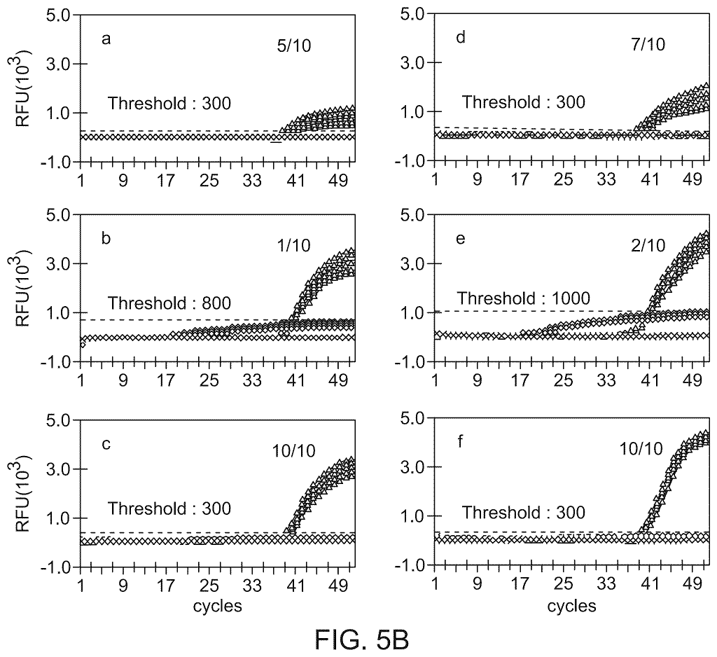

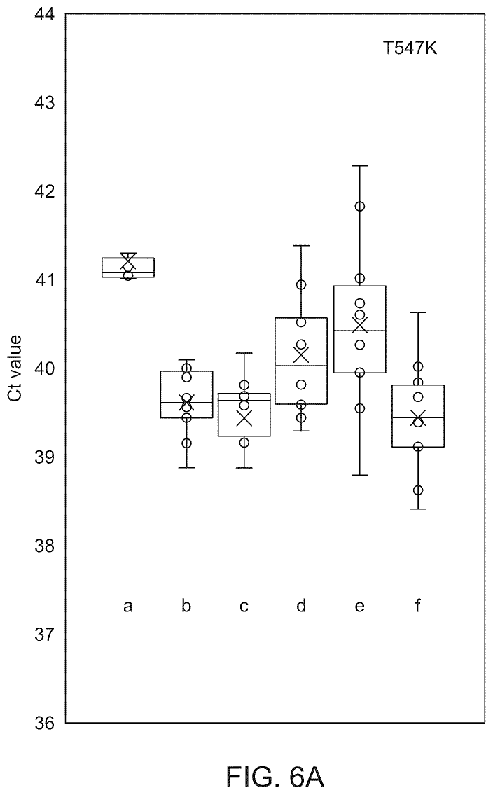

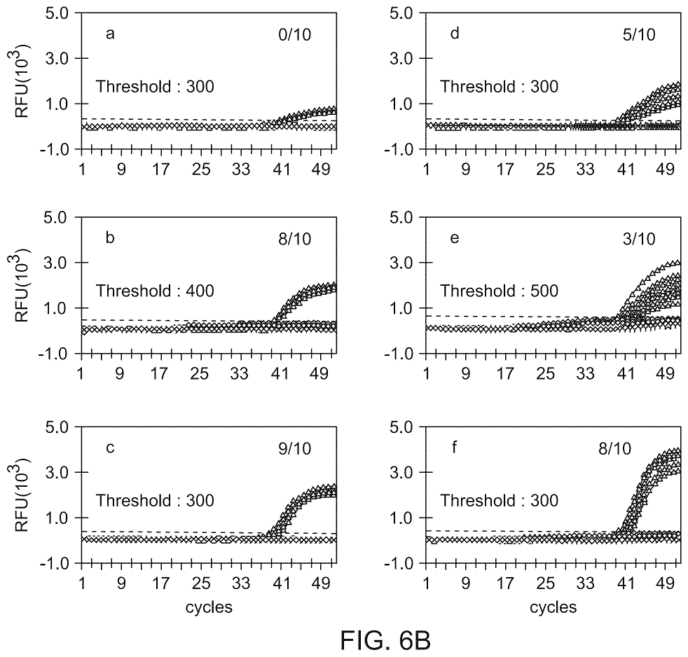

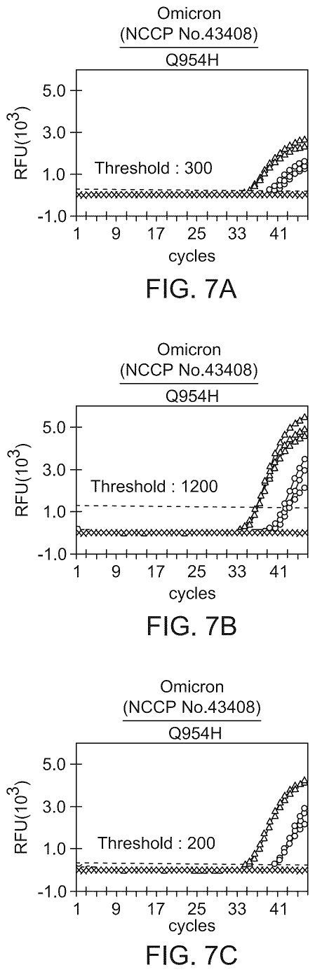

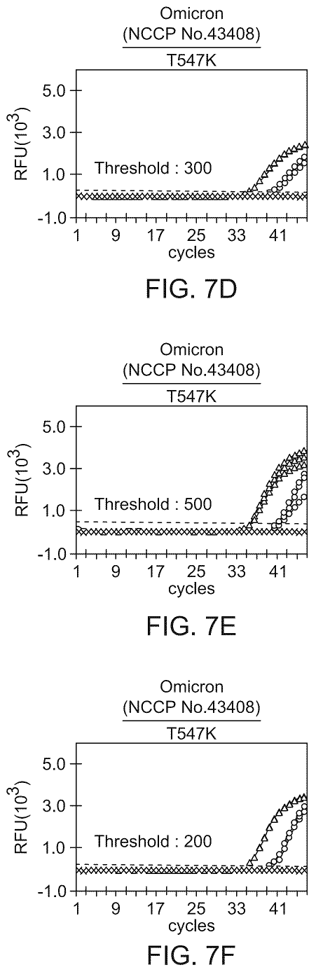

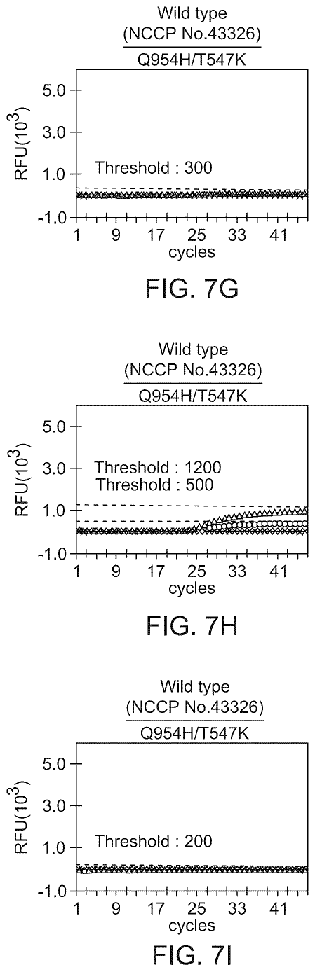

In FIG. 2, b, e, and h are test groups using 0.5 μM of the Com probe: c, f, and i are test groups using 1.0 μM of the Com probe; and a, d, and g are Com probe-free control groups. At this time, the type and the amount of template DNA for each target mutation and the qPCR conditions are the same as shown in FIG. 1. A standard plasmid was used as a template, -Δ- denotes a variant test group with a copy number of 5×10³, -o- denotes a variant test group with a copy number of 5×10², and -□- denotes a variant test group with a copy number of 5×10¹, -⋄- denotes a non-variant wild-type control group with a copy number of 5×10⁷, and -X- denotes a template-free control group.

FIG. 3 shows an effect of inhibiting non-specific PCR signals according to the increase of the Tm of the Com probe.

FAM-Cov19-Q954H-3 was used at 0.75 μM to detect Q954H, and tests were performed without adding a Com probe (a) and using Cov19-Q954-Com-1 (b) and Cov19-Q954-Com-2 (c) each at 0.5 μM. Other qPCR conditions are the same as FIG. 1. A standard plasmid was used as a template, -Δ- denotes a variant test group with a copy number of 5×10³, -o- denotes a variant test group with a copy number of 5×10², and -□- denotes a variant test group with a copy number of 5×10¹, -⋄- denotes a non-variant wild-type control group with a copy number of 5×10⁷, and -X- denotes a template-free control group.

FIG. 4 is a schematic diagram illustrating the principle of controlling qPCR non-specific signals from wild-type samples when a Com probe is added.

FIG. 4A explains qPCR signals generated from the Com probe-free test group. The qPCR diagram on the right shows an example of a test result without the addition of the Com probe, and is a logarithmic function graph of the qPCR in a of FIG. 3, in which a represents a variant test group with a copy number of 5×10³, and b represents a non-variant wild-type test group with a copy number of 5×10⁷.

FIG. 4B is a diagram explaining the principle of inhibiting qPCR non-specific signals by adding the Com probe. The qPCR diagram on the right shows an example of a test result with the addition of the Com probe, and is a logarithmic function graph of the qPCR in c of FIG. 3, in which a represents a variant test group with a copy number of 5×10³, and b represents a non-variant wild-type test group with a copy number of 5×10⁷.

FIG. 5 shows the deviation of the Ct value under each qPCR condition (A) and the results of repeated Q954H detection tests (B) for setting the threshold.

-Δ- denotes a test group using pCov19-S-Q954 (variant) with a copy number of 3×10¹, and -⋄- denotes a test group using pCov19-S-H954 (non-variant) with a copy number of 1×10⁷. -X-denotes a template-free control group. The test was repeatedly performed ten times using a fluorescent hydrolysis probe and the Com probe as shown in Table 6. Table 6 shows the Ct value (A) and the derived qPCR thresholds and positive detections (B) of each qPCR test.

FIG. 6 shows the deviation of the Ct value under each qPCR condition (A) and the results of repeated T547K detection tests (B) for setting the threshold.

-Δ- denotes a test group using pCov19-S-T547 (variant) with a copy number of 3×10¹, and -⋄- denotes a test group using pCov19-S-K547 (non-variant) with a copy number of 1×10⁷. -X- denotes a template-free control group. The test was repeatedly performed ten times using a fluorescent hydrolysis probe and the Com probe as shown in Table 6. Table 7 shows the Ct value (A) and the derived qPCR thresholds and positive detections (B) of each qPCR test.

FIG. 7 shows the results of RT-qPCR using SARS-Cov2 RNA samples.

As fluorescent hydrolysis probes, FAM-Cov19-Q954H (a), FAM-Cov19-Q954H-3 (b and d), JOE-Cov19-T547K-1 (d), and JOE-Cov-T547K-5 (e and f) were used, and as the Com probes, Cov19-Q954-Com-2 (c) and Cov19-T547-Com-3 (f) were used. For each reaction, NCCP No. 43408 RNA was used in an amount corresponding to copy numbers of 2×10⁴ (-Δ-) and 2×10³ (-o-). Detection tests for Q954H (-▴-) and T547K (-•-) using NCCP No. 43326 RNA were performed with a copy number of 2×10⁷.

### MODES OF THE INVENTION

The present invention relates to a quantitative polymerase chain reaction (qPCR) kit for variant detection, including:
(a) at least one template including a target base sequence in which a position with a high probability of mutation is known;
(b) at least one forward primer and at least one reverse primer for the template;
(c) a nucleic acid polymerase polymerizing a nucleic acid from the forward primer and the reverse primer;
(d) at least one fluorescent hydrolysis probe for distinguishing a variant, which is complementary to a variant target base sequence and binds to the variant target base sequence; and
(e) at least one Com probe for inhibiting non-specific signals from a non-variant target base sequence:
wherein the Com probe for inhibiting non-specific signals is a probe which is complementary to the non-variant target base sequence, preferentially binds to the non-variant target base sequence rather than the variant target base sequence, and has a structure in which a 3′ end is not extended by the nucleic acid polymerase.

In addition, the present invention relates to a qPCR kit in which a fluorescent hydrolysis probe is a probe which is complementary to the variant target base sequence, consists of 10 to 30 nucleotides, and of which a 5′ end and a 3′ end are modified with a reporter and a quencher capable of transferring fluorescence resonance energy, respectively.

In addition, the present invention relates to a qPCR kit in which the Com probe for inhibiting non-specific signals consists of 10 to 40 nucleotides complementary to the non-variant target base sequence and does not include a fluorescence agent or coloring agent or is not linked to a fluorescence agent or coloring agent.

In addition, the present invention relates to a qPCR kit in which the Com probe for inhibiting non-specific signals has the same number of bases as the fluorescent hydrolysis probe or is 1 to 10 bases longer.

In addition, the present invention relates to a qPCR kit in which the Com probe for inhibiting non-specific signals has a Tm equal to or higher than the Tm value of the fluorescent hydrolysis probe.

In addition, the present invention relates to a qPCR kit in which the Com probe for inhibiting non-specific signals has a structure that prevents extension by a nucleic acid polymerase at a 3′ end. The structure that prevents extension by a nucleic acid polymerase at a 3′ end may be a structure that a 3′ end of the Com probe is bound to or modified with a 3′ terminal modifier, but it is not limited to a structure that is bound to or modified with a 3′ terminal modifier. In addition, the 3′ terminal modifier is not particularly limited, but examples thereof may include 3′-Phosphate, 3′-Spacer C3, 3′-ddC, and 3′-Inverted End.

In addition, the present invention relates to a Com probe for inhibiting non-specific signals, which is used in a quantitative polymerase chain reaction (qPCR) method or kit for variant detection, including:
(a) at least one template including a target base sequence in which a position with a high probability of mutation is known;
(b) at least one forward primer and at least one reverse primer for the template;
(c) a DNA polymerase polymerizing DNA from the forward primer and the reverse primer; and
(d) at least one fluorescent hydrolysis probe for distinguishing a variant, which is complementary to a variant target base sequence and binds to the variant target base sequence,
the Com probe being complementary to the non-variant target base sequence, preferentially binding to the non-variant target base sequence rather than the variant target base sequence, having a structure in which a 3′ end is not extended by the DNA polymerase, and inhibiting non-specific signals from a non-variant target base sequence, thereby improving the specificity of variant detection.

In addition, according to the present invention, the Com probe for inhibiting non-specific signals consists of 10 to 40 nucleotides complementary to the non-variant target base sequence and does not include a fluorescence agent or coloring agent or is not linked to a fluorescence agent or coloring agent.

In addition, according to the present invention, the Com probe for inhibiting non-specific signals has the same number of bases as the fluorescent hydrolysis probe or is 1 to 10 bases longer.

In addition, according to the present invention, the Com probe for inhibiting non-specific signals has a Tm equal to or higher than the Tm value of the fluorescent hydrolysis probe.

In addition, the present invention relates to a Com probe for inhibiting non-specific signals having a structure that prevents extension by a nucleic acid polymerase at a 3′ end of the Com probe for inhibiting non-specific signals. The structure that prevents extension by a nucleic acid polymerase at a 3′ end of the Com probe may be a structure that a 3′ end of the Com probe is bound to or modified with a 3′ terminal modifier, but it is not limited to a structure that is bound to or modified with a 3′ terminal modifier. In addition, the 3′ terminal modifier is not particularly limited, but examples thereof may include 3′-Phosphate, 3′-Spacer C3, 3′-ddC, and 3′-Inverted End.

In addition, the present invention relates to a Com probe for inhibiting non-specific signals, wherein the fluorescent hydrolysis probe is a probe which is complementary to the variant target base sequence, consists of 10 to 30 nucleotides, and of which a 5′ end and a 3′ end are modified with a reporter and a quencher capable of transferring fluorescence resonance energy, respectively.

In addition, the present invention relates to a method of detecting a genetic variant with high specificity, including:
(A) adding a Com probe for inhibiting non-specific signals which is complementary to the non-variant target base sequence, preferentially binds to the non-variant target base sequence rather than the variant target base sequence, and has a structure in which a 3′ end is not extended by a nucleic acid polymerase to a quantitative polymerase chain reaction (qPCR) solution for variant detection containing one or more templates including a target base sequence in which a position with a high probability of mutation is known: a forward primer and a reverse primer for the template: a nucleic acid polymerase polymerizing a nucleic acid from the forward primer and the reverse primer; and a fluorescent hydrolysis probe for distinguishing a variant, which is complementary to a variant target base sequence and binds to the variant target base sequence; and
(B) performing PCR after (A).

In addition, the present invention relates to a method of detecting a genetic variant, further including:
(C) obtaining an amplification curve from the reaction in (B); and
(D) determining, from the amplification curve, whether the target base sequence includes a mutation.

In addition, the present invention relates to a method of detecting a genetic variant in which the forward primer has one or more bases at a 3′ end corresponding to a position with a high probability of mutation in the target base sequence.

In addition, the present invention relates to a method of detecting a genetic variant in which the mutation is a single nucleotide polymorphism.

In addition, the present invention relates to a method of detecting a genetic variant in which the nucleic acid polymerase is a thermostable DNA polymerase.

In addition, the present invention relates to a method of detecting a genetic variant in which the nucleic acid polymerase is a wild-type or variant DNA polymerase.

In addition, the present invention relates to a method of detecting a genetic variant in which the Com probe for inhibiting non-specific signals consists of 10 to 40 nucleotides.

In addition, the present invention relates to a method of detecting a genetic variant in which the Com probe for inhibiting non-specific signals has the same number of bases as the fluorescent hydrolysis probe or is 1 to 10 bases longer.

In addition, the present invention relates to a method of detecting a genetic variant in which the Com probe for inhibiting non-specific signals has a Tm equal to or higher than the Tm value of the fluorescent hydrolysis probe.

In addition, the present invention relates to a method of detecting a genetic variant in which the Com probe for inhibiting non-specific signals has a structure that prevents extension by a nucleic acid polymerase at a 3′ end. The structure that prevents extension by a nucleic acid polymerase at a 3′ end of the Com probe may be a structure that a 3′ end of the Com probe is bound to or modified with a 3′ terminal modifier, but it is not limited to a structure that is bound to or modified with a 3′ terminal modifier. In addition, the 3′ terminal modifier is not particularly limited, but examples thereof may include 3′-Phosphate, 3′-Spacer C3, 3′-ddC, and 3′-Inverted End.

In addition, the present invention relates to a method of detecting a genetic variant in which the fluorescent hydrolysis probe is a probe that is complementary to the variant target base sequence, consists of 10 to 30 nucleotides, and of which a 5′ end and a 3′ end are modified with a reporter and a quencher capable of transferring fluorescence resonance energy, respectively.

In addition, the present invention relates to a Com probe that improves the specificity and sensitivity of polymerase chain reaction (PCR) for detecting a SARS-Cov-2 variant, in which the Com probe is one or more selected from:
1) a Com probe including an amino acid sequence of TGGTCAACCAAAATGCACAAG (SEQ ID NO: 12) and having a structure that prevents extension by a nucleic acid polymerase at a 3′ end;
2) a Com probe including an amino acid sequence of GTGGTCAACCAAAATGCACAAGC (SEQ ID NO: 13) and having a structure that prevents extension by a nucleic acid polymerase at a 3′ end;
3) a Com probe including an amino acid sequence of TCAATGGTTTAACAGGCACAGGTG (SEQ ID NO: 19) and having a structure that prevents extension by a nucleic acid polymerase at a 3′ end; and
4) a Com probe including an amino acid sequence of GTGTGTTAATCTTACAACCAGAACTCA (SEQ ID NO: 25) and having a structure that prevents extension by a nucleic acid polymerase at a 3′ end. The structure that prevents extension by a nucleic acid polymerase at a 3′ end of the Com probe may be a structure that a 3′ end of the Com probe is bound to or modified with a 3′ terminal modifier, but it is not limited to a structure that is bound to or modified with a 3′ terminal modifier. In addition, the 3′ terminal modifier is not particularly limited, but examples thereof may include 3′-Phosphate, 3′-Spacer C3, 3′-ddC, and 3′-Inverted End.

In addition, the present invention relates to a polymerase chain reaction (PCR) kit for detecting a SARS-Cov-2 variant, including the Com probe.

In addition, the present invention relates to a method of detecting a SARS-Cov-2 variant from a sample with high specificity and sensitivity using the polymerase chain reaction (PCR) kit for detecting a SARS-Cov-2 variant.

Hereinafter, the features of the present invention will be described in more detail through specific examples and test examples. However, it is obvious to those skilled in the art that the scope of the present invention is not limited to the scope described in the examples and test examples.

### SARS-Cov-2 RNA Samples

The SARS-Cov-2 RNA samples were obtained from the National Culture Collection for Pathogens (NCCP), which distributed three standard pathogen resources (NCCP43326, NCCP43410, NCCP43408). Among the RNA samples distributed, NCCP43326 is wild-type Covid 19, NCCP43410 is a delta variant, and NCCP43408 is an omicron variant (Table 1). The RNA samples were frozen and stored at −70° C. before use.

### SARS-Cov-2 Sequence Comparison and Variant Screening

To compare the sequences of the variants, the sequence information of the Spike Trimer coding region among the genomic sequences of each sample registered with the Global Initiative on Sharing All Influenza Data (GISAID) was compared and analyzed, and three mutations, which were S: T19R (21618 C>G), S: T547K (23202 C>A), and S: Q954H (24424 A>T), were selected as regions for distinguishing variants.

Table 1 shows a comparison of the SARS-Cov-2 RNA samples and variant sequences.

TABLE 1

| | | GISAID | Sequence | | |
|---|---|---|---|---|---|
| | | Accession | T19R | T547K | Q954H |
| Variants | NCCP no. | No. | (C > G) | (C > A) | (A > T) |
| Wild | NCCP43326 | EPI_ISL_{—} | C | C | A |
| type | | 407193 | | | |
| (Covid | | | | | |
| 19) | | | | | |
| Omicron | NCCP43408 | EPI_ISL_{—} | C | A | T |
| | | 6959993 | | | |
| Delta | NCCP43410 | EPI_ISL_{—} | G | C | A |
| | | 6744823 | | | |

### Preparation of Standard Plasmid DNA Samples

Using the three types of standard pathogen resource RNA as a template, a PCR product including the T19R, T547K, and Q954H regions of the S gene of SARS-Cov-2 was prepared using the primer set for standard plasmid construction shown in Table 2, and then cloned into the pTOP-TA vector [Enzynomics Co., Ltd., Korea] to construct three wild types (pCov19-S-T19, pCov19-S-T547, pCov19-S-Q954) and 3 variants (pCov19-S-R19, pCov19-S-K547, pCov19-S-H954) as standard plasmid DNA. Each standard plasmid DNA was cleaved with the restriction enzyme NotI, purified, and then diluted with sterile distilled water to prepare 1×10⁸ copies/μL. The DNA prepared in this manner was frozen and stored before being used in experiments.

### Primers/Probes/Com Probes and Standard Plasmids

Table 2 shows information on the primers, probes, Com probes, and plasmids used in the examples and test examples of the present invention.

TABLE 2

| | SEQ ID No., | |
|---|---|---|
| Name | modification (5′-/-3′) (Tm) | Remarks (Target) |
| primer/probe/Com probe | | |
| Cov19-S-09F | SEQ ID NO. 1 | F Primer (T19R) for constructing |
| | | plasmid DNA |
| Cov19-S-09R | SEQ ID NO. 2 | R Primer (T19R) for constructing |
| | | plasmid DNA |
| Cov19-S-04F | SEQ ID NO. 3 | F Primer (T547K) for |
| | | constructing plasmid DNA |
| Cov19-S-04R | SEQ ID NO. 4 | R Primer (T547K) for |
| | | constructing plasmid DNA |
| Cov19-S-954F | SEQ ID NO. 5 | F Primer (Q954H) for |
| | | constructing plasmid DNA |
| Cov19-S-954R | SEQ ID NO. 6 | R Primer (Q954H) for |
| | | constructing plasmid DNA |
| Cov19-Q954H- | SEQ ID NO. 7 | F Primer (Q954H) |
| F1 | | |
| Cov19-Q954H- | SEQ ID NO. 8 | R Primer (Q954H) |
| R1 | | |
| FAM-Cov19- | SEQ ID NO. 9, FAM-/-BHQ1 | Low-Tm Probe (Q954H) |
| Q954H | (51.4° C.) | |
| FAM-Cov19- | SEQ ID NO. 10, FAM-/-BHQ1 | Medium-Tm Probe (Q954H) |
| Q954H-1 | (54.3° C.) | |
| FAM-Cov19- | SEQ ID NO. 11, FAM-/-BHQ1 | High-Tm Probe (Q954H) |
| Q954H-3 | (57.5° C.) | |
| Cov19-Q954- | SEQ ID NO. 12, /-p (57.5° C.) | Com probe (Q954H) |
| Com-1 | | |
| Cov19-Q954- | SEQ ID NO. 13, /-p (62.9° C.) | Com probe (Q954H) |
| Com-2 | | |
| Cov19-T547K-F | SEQ ID NO. 14 | F Primer (T547K) |
| Cov19-T547K- | SEQ ID NO. 15 | R Primer (T547K) |
| R | | |
| JOE-Cov19- | SEQ ID NO. 16, JOE-/-BHQ1 | Low-Tm Probe (T547K) |
| T547K-1 | (52.3° C.) | |
| JOE-Cov19- | SEQ ID NO. 17, JOE-/-BHQ1 | Medium-Tm Probe (T547K) |
| T547K-3 | (55.4° C.)) | |
| JOE-Cov19- | SEQ ID NO. 18, JOE-/-BHQ1 | High-Tm Probe (T547K) |
| T547K-5 | (58.4° C.) | |
| Cov19-T547- | SEQ ID NO. 19, /-p (63.6° C.) | Com probe (T547K) |
| Com-3 | | |
| Cov19-T19R-F | SEQ ID NO. 20 | F Primer (T19R) |
| Cov19-T19R-R | SEQ ID NO. 21 | R Primer (T19R) |
| FAM-Cov19- | SEQ ID NO. 22, FAM-/-BHQ1 | Low-Tm Probe (T19R) |
| T19R-S1 | (54.7° C.) | |
| FAM-Cov19- | SEQ ID NO. 23, FAM-/-BHQ1 | Medium-Tm probe (T19R) |
| T19R-S2 | (57.6° C.) | |
| FAM-Cov19- | SEQ ID NO. 24, FAM-/-BHQ1 | High-Tm Probe (T19R) |
| T19R-S3 | (58.3° C.) | |
| Cov19-T19- | SEQ ID NO. 25, /-p (63.7° C.) | Com probe (T19R) |
| Com-3 | | |

Standard Plasmid

| | | |
|---|---|---|
| pCov19-S-T19 | SEQ ID NO. 26 | Template (wild type, T19) |
| pCov19-S-R19 | SEQ ID NO. 27 | Template (Delta, R19) |
| pCov19-S-T547 | SEQ ID NO. 28 | Template (wild type, T547) |
| pCov19-S-K547 | SEQ ID NO. 29 | Template (Omicron, K547) |
| pCov19-S-Q954 | SEQ ID NO. 30 | Template (wild type, Q954) |
| pCov19-S-H954 | SEQ ID NO. 31 | Template (Omicron, H954) |

Implementation of qPCR

The fluorescent hydrolysis probe was designed with a base sequence capable of hybridizing to the amplified product for real-time qPCR detection, and in order to apply the principle of fluorescence resonance energy transfer (FRET), a probe in which a fluorescent substance (fluorescein amidite (FAM) or 6-carboxy-4′,5′-dichloro-2′,7′-dimethoxyfluorescein succinimidyl ester (JOE)) was bound to a 5′ end and a quencher (BHQ1) was bound to a 3′ end was used. In addition, a phosphate group was attached to the 3′ end of the Com probe to prevent amplification by a polymerase. The oligonucleotides used were produced using the oligonucleotide synthesis system of the applicant, GenoTech Corp.

Using each DNA sample prepared as above as a template, real-time PCR was performed using the primers, fluorescent hydrolysis probes, and Com probes prepared according to the test. Cov19-Q954H-F1 and Cov19-Q954H-R1 as primers for Q954H detection, Cov19-T547K-F and Cov19-T547K-R as primers for T547K detection, and Cov19-T19R-F and Cov19-T19R-R as primers for T19R detection were used by adding at 0.5 to 0.75 μM each. The types and concentrations of the fluorescent probes and Com probes used in other test examples were described in each example. The polymerase and PCR buffer solution used were products from Enzynomics Co. Ltd. (cat no. RT 431M), and the total volume of the reaction solution was adjusted to 20 μL. pCov19-S-T19, pCov19-S-T547, and pCov19-S-Q954 were used as wild-type DNA standards for detection tests, and for variants, pCov19-S-R19, pCov19-S-K547, and pCov19-S-H954 were used in the same amount as in the example, and at this time, qPCR was performed at 95° C. for 10 minutes, followed by 45 to 50 cycles of 10 to 15 seconds at 95° C. and 10 to 15 seconds at 60° C., using the CFX9600 Real-Time System. Quantitative reverse transcription PCR (RT-qPCR) using the three standard pathogen resources (NCCP43326, NCCP43410, NCCP43408) distributed from the NCCP as SARS-Cov-2 RNA standards was performed at 50° C. for 30 minutes and at 95° C. for 10 minutes, followed by 45 to 50 cycles of 10 to 15 seconds at 95° C. and 10 to 15 seconds at 60° C., using the CFX9600 Real-Time System.

### Example 1: Generation of Non-Specific Signals in qPCR Depending on the Fluorescent Probe Length

In the present example, the change in qPCR signals and Ct value according to the length of the fluorescent hydrolysis probe for variant detection was tested. A test was performed to detect T19R, T547K, and Q954H, which are the mutation regions of SARS-Cov-2 as a variant for the detection test. Using each DNA standard plasmid in Table 2 as a template, the test was performed while increasing the Tm value by increasing the bases of the fluorescent probe by 1 to 3 bases (FIG. 1 and Table 3). The templates used in the test were pCov19-S-Q954 (wild-type) and pCov19-S-H954 (variant) for the Q954H variant distinguishing test, pCov19-S-T547 (wild type) and pCov19-S-K547 (variant) for the T547K variant distinguishing test, and Cov19-S-T19 (wild type) and pCov19-S-R19 (variant type) standard plasmids for the T19R variant distinguishing test.

As a result of the test, the use of probes that are 1 to 3 bases longer, such as b, e, and h (using a medium Tm probe) and c, f, and i (using a high Tm probe) in FIG. 1, exhibited higher signals than when using a short specific probe (a, d, and g in FIG. 1: using a low Tm probe), and in particular, in the tests in which a low copy number (5×10¹ copies) was used, the use of a long specific probe exhibited a slightly lower Ct value (Table 3: Ct value of qPCR depending on the increase in Tm of probes for variant detection). However, as a result of using a long probe (high Tm probe) to obtain a high signal and a low Ct value, non-specific signals were exhibited in the non-variant test strain at high concentrations (10⁷ copies/reaction), as shown in c, f, and i of FIG. 1. Therefore, in order to use a long probe (high Tm probe) with a high signal value, the threshold needs to be raised and adjusted according to the test to accurately distinguish variants. However, when the threshold is increased, the overall signal height decreases and in particular, the Ct value increases, so the advantage of using a long probe may be lost. Therefore, a new method is required to inhibit non-specific signals while maintaining high signals when using a long probe.

TABLE 3

| | Ct (threshold, 200)* | | |
|---|---|---|---|
| | Target | | |
| | (5 × 10¹ copies/reaction) | | |
| Probe** | Q954H | T547K | T19R |
| Low Tm probes | 38.08 | 39.20 | 36.80 |
| Medium Tm probes | 38.18 | 38.37 | 36.31 |
| High Tm probes | 37.48 | 37.96 | 36.28 |

*The average Ct value of the qPCR test with a copy number of 5 × 10¹ in FIG. 1 is shown.
**The probes used are fluorescent probes for distinguishing variants as shown in Table 2 and FIG. 1.
The test details are shown in FIG. 1.

### Example 2: Inhibition of Non-Specific qPCR Signals Through Addition of Com Probe

When a variant-specific fluorescent probe with a high Tm is used, a high non-specific signal is generated in a high-concentration wild-type sample (c, f, and i in FIG. 1). This is a non-specific signal that is exhibited when a variant-specific fluorescent probe binds to a high-concentration wild-type sample at a certain level, albeit with low binding affinity (hybridization), and is degraded by a Taq DNA polymerase. As a method to control such non-specific signals, the present inventors developed a Com probe (named “Com probe” after Competition probe or Combination probe), which may bind preferentially to wild-type samples over fluorescent probes.

The Com probe was composed of a non-amplified oligonucleotide (phosphorylated at a 3′-end) of the same sequence as the sequence of the wild type including the same sequence as the region where a variant-specific probe binds.

For specific testing in Example 2, high-Tm probes of FAM-Cov19-Q954H-3 for Q954H detection, JOE-Cov19-T547K-5 for T547K detection, and FAM-Cov19-T19R-S3 for T19R detection were used at 0.75 μM each, and Cov19-Q954-Com-2 for Q954H detection, Cov19-T547-Com-3 for T547K detection, and Cov19-T19-Com-3 for T19R detection were used as Com probes, and the qPCR signal patterns and Ct values of the Com probe-free test group and the test groups to which the Com probe was added at a concentration of 0.5 to 1.0 μM were compared (FIG. 2 and Table 4).

As a result, when the Com probe was not added, a clear non-specific signal was observed (a, d, and g in FIG. 2), but when the Com probe was added, as the Com probe concentration increased, the non-specific signals significantly decreased in the high-concentration wild-type test groups. In particular, even with the addition of 1.0 μM of the Com probe, there was little effect on qPCR, and a distinct non-specific signal reducing effect was observed (b, e, h, c, f, and i in FIG. 2). In particular, excellent qPCR detection ability was exhibited even in the variant detection tests with a small amount (copy number of 5×10¹), and it was confirmed that even when the Com probe was added, the overall Ct value was maintained at a level equivalent to the case where the Com probe was not added (Table 4: qPCR Ct value according to the amount of Com probe used).

TABLE 4

| | | Ct (threshold, 200)* | | | |
|---|---|---|---|---|---|
| | | Mutant | | | Wild type |
| | Com probe | (copies/reaction) | | | (copies/reaction) |
| Target | (μM) | 5 × 10³ | 5 × 10² | 5 × 10¹ | 5 × 10⁷ |
| Q954H | 0 | 30.11 | 33.54 | 37.02 | 16.53 |
| | 0.5 | 30.26 | 33.3 | 36.44 | 0 |
| | 1 | 30.55 | 34.3 | 38.05 | 0 |
| T547K | 0 | 30.95 | 33.88 | 37.65 | 19.09 |
| | 0.5 | 30.81 | 34.12 | 37.47 | 0 |
| | 1 | 31.08 | 34.09 | 37.57 | 0 |
| T19R | 0 | 30.09 | 32.88 | 36.86 | 20.57 |
| | 0.5 | 29.97 | 32.82 | 36.67 | 24.66 |
| | 1 | 30.27 | 32.89 | 36.33 | 0 |

*Average Ct value of the repeated qPCR tests in FIG. 2.

### Example 3: Effect According to the Length of Com Probe

To observe the effect according to the length of the Com probe, the effect of a Com probe (Cov19-Q954-Com-1) that had the same length as the fluorescent probe and a Com probe (Cov19-Q954-Com-2) that was 3 bases longer than the fluorescent probe was compared. At this time, the Com probe was tested at a concentration of 0.5 μM, which was less than the 0.75 μM concentration of the fluorescent probe. As a result, a clear effect of inhibiting the non-specific signals generated when no Com probe was added (a of FIG. 3) was confirmed for the long Com probes rather than the short Com probes (b and c of FIG. 3). At this time, the average Ct value was observed to be the same as that when no Com probe was added, or to decrease from Ct=37.02 without the addition of the Com probe to Ct=36.44 with the addition of the Com probe, as shown in the test results obtained with a copy number of 5×10¹ (Table 5). This decrease in the Ct value may provide favorable conditions for detection of low-concentration samples.

Table 5 below shows the Ct values of qPCR according to the Com probe length.

TABLE 5

| | Ct (threshold, 200)* | | | |
|---|---|---|---|---|
| | Mutant (Q954H) | | | Wild type |
| | (copies/reaction) | | | (copies/reaction) |
| Com probe | 5 × 10³ | 5 × 10³ | 5 × 10³ | 5 × 10³ |
| No add | 30.11 | 33.54 | 37.02 | 16.53 |
| Cov19-Q954- | 29.89 | 33.35 | 37.41 | 20.15 |
| Com-1 (0.5 | | | | |
| μM) | | | | |
| Cov19-Q954- | 30.26 | 33.30 | 36.44 | 0.00 |
| Com-2 (0.5 | | | | |
| μM) | | | | |

*Average Ct value of the qPCR tests in FIG. 1.

The clear control effect of the high non-specific signals due to the high-concentration wild-type samples in the variant detection qPCR test with the addition of the Com probes of Examples 2 and 3 may be explained as shown in FIG. 4. It is not easy for a fluorescent probe to clearly distinguish between a variant template and a non-variant template based on a single base. However, when there are one or more mismatches between a probe and a template, hybridization robustness is weak, so the probe is generally displaced from the template before hydrolysis by the 3′ nuclease activity of the Taq DNA polymerase during the DNA polymerization process. However, in the presence of an excessive amount of non-variant (wild type) template, a part of the fluorescent probe that mismatches the non-variant template is decomposed by the 3′ nuclease of the Taq DNA polymerase, resulting in an excessive amount of non-specific signals as shown in b of FIG. 4. However, when a certain amount of Com probe is added as in the method of the present invention, competitive binding of the Com probe and the probe to the non-variant template occurs. In particular, since the Com probe is complementary to the non-variant template sequence, it matches the non-variant template sequence, so displacement does not occur, preventing the generation of non-specific signals due to hydrolysis of the fluorescent probe (d of FIG. 4). Similarly, even when a Com probe is added, displacement frequently occurs because the Com probe mismatches a variant sequence, but the fluorescent probe matches the variant sequence and is easily hydrolyzed by the 3′ nuclease, so the qPCR signal is clearly confirmed (a and c of FIG. 4).

### Example 4. Threshold and Positive Detection Through Repeated qPCR Tests for Variant Detection

Repeated tests were conducted to more clearly confirm the effect of the Com probe as described in Example 3. To detect the Q954H mutation and the T547K mutation, the change in the Ct value and threshold according to various types of probes with different Tm values, the amount of probe used, and the conditions of using the Com probe were tested by repeating qPCR ten times. The specific test conditions are shown in Table 6 and Table 7, the repeated Q954H test conditions are shown in Table 6, and the repeated T547K test conditions are shown in Table 7. First, under each test condition, the test with the wild-type standard plasmid was repeated ten times with a copy number of 1×10⁷, and the threshold value of qPCR was determined based on the test results. qPCR that exceeded the determined threshold for each test group and satisfied Ct≤40 was defined as positive detection.

When a low-Tm probe (FAM-Cov19-Q954H) was used at a low concentration of 0.25 μM to detect the Q954H mutation, the number of positive detections was as low as five (test group a in FIG. 5B and Table 6), and when the concentration of the low-Tm probe was increased to 0.5 μM, the number of positive detections slightly increased to seven (test group d in FIG. 5B and Table 6). At this time, the change in the threshold due to the increase in the usage amount was less than 300 in each group, which was not significant. A high-Tm probe (FAM-Cov19-Q954H-3) was used to improve positive detection. However, the number of positive detections was not significantly improved in both the 0.25 μM and 0.5 μM test groups due to the high thresholds of 800 and 1000 (test groups b and e in FIG. 5B and Table 6). However, when the high-Tm probe (FAM-Cov19-Q954H-3) was used, the deviation of the Ct value was reduced compared to the case when the low-Tm probe (FAM-Cov19-Q954H) was used, the Ct value was also smaller, and the signal height was higher (test groups b and e in FIGS. 5A and 5B). Considering these results, positive detection may be greatly improved when applying a method of lowering the high threshold, which is the cause of the low number of positive detections in qPCR using a high-Tm probe.

Therefore, the present inventors performed tests by applying a Com probe capable of lowering the threshold value to the test groups using a high-Tm probe. As predicted, when 0.5 μM of the Com probe (Cov19-Q954-Com-2) was added, it was possible to set the threshold below 300 for both test groups using the high-Tm probe (FAM-Cov19-Q954H-3) at 0.25 μM and 0.5 μM. Due to this low threshold setting, the number of positive detections increased despite the application of the high-Tm probe, and also, fast Ct values and small deviations in the Ct values, which are characteristics of the high-Tm probe, were found (test groups c and f in FIG. 4 and Table 6).

As in the Q954H mutation detection test, as a result of applying a similar high-Tm probe and a Com probe to the T547K mutation detection test, the threshold was lowered, faster Ct values, small Ct value deviations, and high positive detection results were obtained (FIG. 6 and Table 7).

Table 6 shows the repeated test conditions and qPCR results for Q954 mutation detection.

TABLE 6

| Test conditions* (10 cycles) | | a | b | c | d | e | f |
|---|---|---|---|---|---|---|---|
| Probe | FAM-Cov19-Q954H | 0.25 | — | — | 0.5 | — | — |
| | (μM) | | | | | | |
| | FAM-Cov19- | — | 0.25 | 0.25 | — | 0.5 | 0.5 |
| | Q954H-3 (μM) | | | | | | |
| Com probe | Cov19-Q954-Com-2 | | | 0.5 | — | — | 0.5 |
| | (μM) | | | | | | |
| qPCR Result** | Determined | 300 | 800 | 300 | 300 | 1000 | 300 |
| | threshold | | | | | | |
| | Average Ct | 40.86 | 40.41 | 38.94 | 39.51 | 40.39 | 38.84 |
| | Positive/Test (Ct ≤ 40) | 5/10 | 1/10 | 10/10 | 7/10 | 2/10 | 10/10 |

*The test conditions and the used amount of the probes and Com probes in FIG. 5 are shown.
**The threshold determined in the test in FIG. 5 and the average Ct value and number of positive detections of the 10 repeated qPCR tests are shown. When the Ct value was 40 or less, the detection was determined to be a positive detection.

Table 7 shows the repeated test conditions and qPCR results for T547K mutation detection.

TABLE 7

| Test conditions* (10 cycles) | | a | b | c | d | e | f |
|---|---|---|---|---|---|---|---|
| Probe | JOE-Cov19-T547K-1 | 0.25 | — | — | 0.5 | — | — |
| | (μM) | | | | | | |
| | JOE-Cov19-T547K-5 | — | 0.25 | 0.25 | — | 0.5 | 0.5 |
| | (μM) | | | | | | |
| Com probe | Cov19-T547-Com-3 | | | 0.5 | — | — | 0.5 |
| | (μM) | | | | | | |
| qPCR Result** | Determined threshold | 300 | 400 | 300 | 300 | 500 | 300 |
| | Average Ct | 41.23 | 39.61 | 39.44 | 40.14 | 40.51 | 39.43 |
| | Positive/Test (Ct ≤ 40) | 0/10 | 8/10 | 9/10 | 5/10 | 3/10 | 8/10 |

*The test conditions and the used amount of the probes and Com probes in FIG. 6 are shown.
**The threshold determined in the test in FIG. 6 and the average Ct value and number of positive detections of the 10 repeated qPCR tests are shown. When the Ct value was 40 or less, the detection was determined to be a positive detection.

### Example 5: RT-qPCR Test Using SARS-Cov2 RNA Samples

According to the above Examples, the Com probe that strengthens the advantage of exhibiting low Ct values, small Ct value deviations, and high signals through the application of the high-Tm probe and increases a positive detection rate by lowering the threshold was evaluated through RT-qPCR by applying it directly to SARS-Cov-2 RNA samples. As the RNA samples, the RNA of the omicron variant NCCP No. 43408 and the RNA of the wild-type NCCP No. 43326 were used. As the probes, FAM-Cov19-Q954H was used as a low-Tm probe and FAM-Cov19-Q954H-3 was used as a high-Tm probe for Q954H detection at 0.5 μM each, and JOE-Cov19-T547K-1 was used as a low-Tm probe and JOE-Cov19-T547K-5 was used as a high-Tm probe for T547K detection at 0.5 μM each. As the Com probes, Cov19-Q954-Com-2 was used for Q954H detection, and Cov19-T547-Com-3 was used for T547K detection, at 0.35 μM each. At this time, the primer set was used at 0.5 μM each in the test, and the other test conditions were the same as the RT-qPCR test described in the main text.

First, the threshold using the NCCP No. 43326 RNA sample, which was estimated to have a copy number of 2×10⁷, increased to 1200 RFU (Q954H) and 500 RFU (T547K) when a high-Tm probe was applied and decreased to less than 200 RFU when a Com probe was applied together (g, h, and i of FIG. 7). As a result of applying this to perform RT-qPCR for variant detection of each of Q954H and T547K, an excellent improvement in positive detection was found when a high-Tm probe and a Com probe were applied, as in the test using the standard plasmid DNA (Example 4). In particular, the decrease in Ct value was noticeable in the low-concentration RNA samples, and positive detection also became clearer (test groups c and f in FIG. 7 and Table 8).

Table 8 shows the results of the RT-qPCR test using SARS-Cov2 RNA samples.

TABLE 8

| | | Target mutation | | | | | |
|---|---|---|---|---|---|---|---|
| | | Q954H | | | T547K | | |
| | | Test set | | | | | |
| | | a | b | c | d | e | f |
| | | Determined threshold | | | | | |
| Copies/reaction | | 300 | 1200 | 200 | 300 | 500 | 200 |
| 2 × 10⁴ | Average Ct | 35.24 | 36.51 | 34.05 | 35.32 | 35.50 | 34.19 |
| | Positive/Test | 3/3 | 3/3 | 3/3 | 3/3 | 3/3 | 3/3 |
| | (Ct ≤ 40) | | | | | | |
| 2 × 10³ | Average Ct | 40.25 | 42.51 | 39.58 | 40.15 | 41.46 | 39.38 |
| | Positive/Test | 1/3 | 0/3 | 3/3 | 1/3 | 0/3 | 3/3 |
| | (Ct ≤ 40) | | | | | | |

*The conditions of all tests are the same as in FIG. 7. The Ct value shown is the average value, and when the Ct value was 40 or less, the detection was determined to be a positive detection.

### Industrial Applicability

The present invention is very useful in detecting genetic variants in various fields including health, medicine, pharmacy, veterinary medicine, and food fields.

### Sequence List Pretext

An electronic file including a sequence list is attached.

1-26. (canceled)

27. A Com probe for inhibiting non-specific signals, which is used in a quantitative polymerase chain reaction (qPCR) method or kit for variant detection, comprising:
(a) at least one forward primer for a template including a target base sequence in which a position with a high probability of mutation is known;
(b) at least one reverse primer for the template;
(c) a DNA polymerase that polymerizes DNA from the forward primer and the reverse primer; and
(d) at least one fluorescent hydrolysis probe for distinguishing a variant, which is complementary to a variant target base sequence and binds to the variant target base sequence,
wherein the Com probe being complementary to the non-variant target base sequence, preferentially binding to the non-variant target base sequence rather than the variant target base sequence, having a structure in which a 3′ end is not extended by the DNA polymerase, and inhibiting non-specific signals from a non-variant target base sequence, thereby improving the specificity of variant detection.

28. The Com probe of claim 27, wherein the Com probe for inhibiting non-specific signals consists of 10 to 40 nucleotides complementary to the non-variant target base sequence and does not include a fluorescence agent or coloring agent.

29. The Com probe of claim 27, wherein the Corn probe for inhibiting non-specific signals has a Tm equal to or higher than the Tm value of the fluorescent hydrolysis probe.

30. The Com probe of claim 27, wherein the Corn probe for inhibiting non-specific signals has the same number of bases as the fluorescent hydrolysis probe or is 1 to 10 bases longer.

31. The Com probe of claim 27, wherein a 3′ end of the Com probe for inhibiting non-specific signals has a structure that is bound to or modified with a 3′ terminal modifier to prevent extension by the nucleic acid polymerase.

32. The Corn probe of claim 27, wherein the fluorescent hydrolysis probe is a probe which is complementary to the variant target base sequence, consists of 10 to 30 nucleotides, and of which a 5′ end and a 3′ end are modified with a reporter and a quencher capable of transferring fluorescence resonance energy, respectively.

33. A quantitative polymerase chain reaction (qPCR) kit for variant detection, comprising:
(a) at least one forward primer for a template including a target base sequence in which a position with a high probability of mutation is known;
(b) at least one reverse primer for the template;
(c) a nucleic acid polymerase that polymerizes a nucleic acid from the forward primer and the reverse primer;
(d) at least one fluorescent hydrolysis probe for distinguishing a variant, which is complementary to a variant target base sequence and binds to the variant target base sequence; and
(e) at least one Com probe for inhibiting non-specific signals from a non-variant target base sequence;
wherein the Com probe for inhibiting non-specific signals is a probe which is complementary to the non-variant target base sequence, preferentially binds to the non-variant target base sequence rather than the variant target base sequence, and has a structure in which a 3′ end is not extended by the nucleic acid polymerase.

34. The qPCR kit of claim 33,
(i) wherein the fluorescent hydrolysis probe is a probe which is complementary to the variant target base sequence, consists of 10 to 30 nucleotides, and of which a 5′ end and a 3′ end are modified with a reporter and a quencher capable of transferring fluorescence resonance energy, respectively;
(ii wherein the Corn probe for inhibiting non-specific signals consists of 10 to 40 nucleotides complementary to the non-variant target base sequence and does not include a fluorescence agent or coloring agent;
(iii) wherein the Corn probe for inhibiting non-specific signals has a Tm equal to or higher than the Tm value of the fluorescent hydrolysis probe;
(iv) wherein the Corn probe for inhibiting non-specific signals has the same number of bases as the fluorescent hydrolysis probe or is 1 to 10 bases longer; and/or
(v) wherein the Com probe for inhibiting non-specific signals has a structure that is bound to or modified with a 3′ terminal modifier to prevent extension by a nucleic acid polymerase at a 3′ end.

35. A method of detecting a genetic variant with high specificity, comprising:
(A) adding a Corn probe to a quantitative polymerase chain reaction (qPCR) solution for variant detection:
wherein the Corn probe inhibits non-specific signals, is complementary to the non-variant target base sequence, preferentially binds to the non-variant target base sequence rather than the variant target base sequence, and has a structure where the 3′ end is not extended by a nucleic acid polymerase, and
wherein the qPCR solution comprises:
(i) at least one template including a target base sequence with a position of high mutation probability,
(ii) at least one forward primer and at least one reverse primer, wherein the forward primer has one or more bases at its 3′ end corresponding to the high-probability mutation position in the target base sequence,
(iii) a nucleic acid polymerase that polymerizes nucleic acids from the forward and reverse primer; and
(iv) at least one fluorescent hydrolysis probe complementary to a variant target base sequence for distinguishing a variant, and
(B) performing PCR after step (A).

36. The method of claim 35, further comprising:
(C) obtaining an amplification curve from the reaction in (B); and
(D) determining, from the amplification curve, whether the target base sequence includes a mutation.

37. The method of claim 35, wherein the mutation is a single nucleotide polymorphism.

38. The method of claim 35, wherein the nucleic acid polymerase is a thermostable DNA polymerase.

39. The method of claim 35, wherein the nucleic acid polymerase is a wild-type or variant DNA polymerase.

40. The method of claim 35,
(i) wherein the Com probe for inhibiting non-specific signals consists of 10 to 40 nucleotides;
(ii) wherein the Com probe for inhibiting non-specific signals has a Tm equal to or higher than the Tm value of the fluorescent hydrolysis probe;
(iii) wherein a 3′ end of the Com probe for inhibiting non-specific signals has a structure that is bound to or modified with a 3′ terminal modifier to prevent extension by the nucleic acid polymerase; and/or
(iv) wherein the Com probe for inhibiting non-specific signals has the same number of bases as the fluorescent hydrolysis probe or is 1 to 10 bases longer.

41. The method of claim 35, wherein the fluorescent hydrolysis probe is a probe which is complementary to the variant target base sequence, consists of 10 to 30 nucleotides, and of which a 5′ end and a 3′ end are modified with a reporter and a quencher capable of transferring fluorescence resonance energy, respectively.

42. A Com probe that improves the specificity and sensitivity of polymerase chain reaction (PCR) for detecting a SARS-Cov-2 variant, wherein the Corn probe is at least one selected from:
1) a Corn probe comprising the nucleotide sequence of TGGTCAACCAAAATGCACAAG (SEQ ID NO: 12) and having a structure that inhibits extension by a nucleic acid polymerase at the 3′ end;
2) a Corn probe comprising the nucleotide sequence of GTGGTCAACCAAAATGCACAAGC (SEQ ID NO: 13) and having a structure that inhibits extension by a nucleic acid polymerase at the 3′ end;
3) a Corn probe comprising the nucleotide sequence of TCAATGGTTTAACAGGCACAGGTG (SEQ ID NO: 19) and having a structure that inhibits extension by a nucleic acid polymerase at the 3′ end; and
4) a Corn probe comprising the nucleotide sequence of GTGTGTTAATCTTACAACCAGAACTCA (SEQ ID NO: 25) and having a structure that inhibits extension by a nucleic acid polymerase at the 3′ end.

43. A polymerase chain reaction (PCR) kit for detecting a SARS-Cov-2 variant, comprising the Corn probe that improves the specificity and sensitivity of PCR for detecting a SARS-Cov-2 variant of claim 42.

44. A method of detecting a SARS-Cov-2 variant from a sample by using the polymerase chain reaction (PCR) kit of claim 43.


SEQUENCE LISTING (31 sequences)

SEQ ID NO: 1 - DNA, 23 bp, synthetic construct
  source 1..23: mol_type = other DNA
gatggttatgtcatgcatgcaaa

SEQ ID NO: 2 - DNA, 23 bp, synthetic construct
  source 1..23: mol_type = other DNA
agtagggactgggtcttcgaatc

SEQ ID NO: 3 - DNA, 23 bp, synthetic construct
  source 1..23: mol_type = other DNA
ccgcatcattttccacttttaag

SEQ ID NO: 4 - DNA, 23 bp, synthetic construct
  source 1..23: mol_type = other DNA
tttgtgggtatggcaatagagtt

SEQ ID NO: 5 - DNA, 22 bp, synthetic construct
  source 1..22: mol_type = other DNA
gcgggtacaatcacttctggtt

SEQ ID NO: 6 - DNA, 23 bp, synthetic construct
  source 1..23: mol_type = other DNA
gaagctctgatttctgcagctct

SEQ ID NO: 7 - DNA, 26 bp, synthetic construct
  source 1..26: mol_type = other DNA
ttggcaaaattcaagactcactttct

SEQ ID NO: 8 - DNA, 24 bp, synthetic construct
  source 1..24: mol_type = other DNA
gagctaagttgtttaacaagcgtg

SEQ ID NO: 9 - DNA, 18 bp, synthetic construct
  source 1..18: mol_type = other DNA
  misc_feature 18: note = BHQ1 quencher
tggtcaaccataatgcac

SEQ ID NO: 10 - DNA, 20 bp, synthetic construct
  source 1..20: mol_type = other DNA
  misc_feature 20: note = BHQ1 quencher
tggtcaaccataatgcacaa

SEQ ID NO: 11 - DNA, 21 bp, synthetic construct
  source 1..21: mol_type = other DNA
  misc_feature 21: note = BHQ1 quencher
tggtcaaccataatgcacaag

SEQ ID NO: 12 - DNA, 21 bp, synthetic construct
  source 1..21: mol_type = other DNA
tggtcaaccaaaatgcacaag

SEQ ID NO: 13 - DNA, 23 bp, synthetic construct
  source 1..23: mol_type = other DNA
gtggtcaaccaaaatgcacaagc

SEQ ID NO: 14 - DNA, 29 bp, synthetic construct
  source 1..29: mol_type = other DNA
gtctactaatttggttaaaaacaaatgtg

SEQ ID NO: 15 - DNA, 26 bp, synthetic construct
  source 1..26: mol_type = other DNA
ttggaaaggcagaaactttttgttag

SEQ ID NO: 16 - DNA, 20 bp, synthetic construct
  source 1..20: mol_type = other DNA
  misc_feature 20: note = BHQ1 quencher
tcaatggtttaaaaggcaca

SEQ ID NO: 17 - DNA, 21 bp, synthetic construct
  source 1..21: mol_type = other DNA
  misc_feature 21: note = BHQ1 quencher
tcaatggtttaaaaggcacag

SEQ ID NO: 18 - DNA, 22 bp, synthetic construct
  source 1..22: mol_type = other DNA
  misc_feature 22: note = BHQ1 quencher
tcaatggtttaaaaggcacagg

SEQ ID NO: 19 - DNA, 24 bp, synthetic construct
  source 1..24: mol_type = other DNA
tcaatggtttaacaggcacaggtg

SEQ ID NO: 20 - DNA, 26 bp, synthetic construct
  source 1..26: mol_type = other DNA
tgtttttcttgttttattgccactag

SEQ ID NO: 21 - DNA, 27 bp, synthetic construct
  source 1..27: mol_type = other DNA
tgaaaactttgtcagggtaataaacac

SEQ ID NO: 22 - DNA, 22 bp, synthetic construct
  source 1..22: mol_type = other DNA
  misc_difference 22: note = BHQ1 quencher
tgtgttaatcttagaaccagaa

SEQ ID NO: 23 - DNA, 23 bp, synthetic construct
  source 1..23: mol_type = other DNA
  misc_feature 23: note = BHQ1 quencher
tgtgttaatcttagaaccagaac

SEQ ID NO: 24 - DNA, 24 bp, synthetic construct
  source 1..24: mol_type = other DNA
  misc_feature 24: note = BHQ1 quencher
tgtgttaatcttagaaccagaact

SEQ ID NO: 25 - DNA, 27 bp, synthetic construct
  source 1..27: mol_type = other DNA
gtgtgttaatcttacaaccagaactca

SEQ ID NO: 26 - DNA, 600 bp, synthetic construct
  source 1..600: mol_type = other DNA
gatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctact

SEQ ID NO: 27 - DNA, 600 bp, synthetic construct
  source 1..600: mol_type = other DNA
gatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttagaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctact

SEQ ID NO: 28 - DNA, 1039 bp, synthetic construct
  source 1..1039: mol_type = other DNA
ccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaa

SEQ ID NO: 29 - DNA, 1039 bp, synthetic construct
  source 1..1039: mol_type = other DNA
ccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaaaaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaa

SEQ ID NO: 30 - DNA, 428 bp, synthetic construct
  source 1..428: mol_type = other DNA
gcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttc

SEQ ID NO: 31 - DNA, 428 bp, synthetic construct
  source 1..428: mol_type = other DNA
gcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccataatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttc